(19) United States
(12) Patent Application Publication
(10) Pub. No.: US 2021/0196846 A1
(43) Pub. Date: Jul. 1, 2021

(54) Self-Contained Fully Automated Non-Incinerating Medical Waste Treatment Device

(71) Applicants: Charles R. Berkeley, Pebble Beach, CA (US); Courtney E. Scott, Pebble Beach, CA (US)

(72) Inventors: Charles R. Berkeley, Pebble Beach, CA (US); Courtney E. Scott, Pebble Beach, CA (US)

(21) Appl. No.: 17/137,050

(22) Filed: Dec. 29, 2020

Related U.S. Application Data
(60) Provisional application No. 62/955,412, filed on Dec. 30, 2019.

(51) Int. Cl. A61L 2/04; A61L 11/00; A61L 2/26; A61L 2/10

(52) U.S. Cl. CPC A61L 2/04; A61L 11/00; A61L 2/26; A61L 2202/14; A61L 2202/122; A61L 2202/20; A61L 2202/121; A61L 2/10

(57) ABSTRACT

The present invention is directed to a non-incinerating medical waste treatment device and methods of use. The device comprising two to four thermal chambers with total independent operation and controls. The desktop mountable device contains electronic, electromechanical, and made-up mechanical assemblies. The intended application of the device and method is for sterilizing medical waste such as bacterial and viral biological contaminated red bag and/or sharps in order to facilitate safe handling and disposal.

### CROSS REFERENCE TO RELATED APPLICATIONS

This application claims the benefit to U.S. Provisional Application No. 62/955,412 filed on Dec. 30, 2019, the entire contents of which are incorporated herein by reference.

### FIELD OF THE INVENTION

This invention relates to a device and method for disposal of biological waste (e.g., medical, infectious, used sharps [needles and scalpels]), and more particularly, to rendering the biological waste safe and sterile by a non-incinerating thermal process. The device is designed to allow for independent processing of medical waste in two or more treatment chambers.

### BACKGROUND OF THE INVENTION

The safe handling and disposal of regulated medical waste from various medical and healthcare facilities is a well-known problem. Of particular concern is the safe collecting and processing of contaminated needles, scalpels, and sharp metal or glass objects, etc., (“sharps”) and other objects such as soft-waste items including gauze, tape, fabrics, and the like, that come into contact with the human body or bodily fluids.

The problems associated with used thermoplastic hypodermic needles and syringes are well known. Collection and disposal of medical waste must be carefully controlled to prevent needlestick injury exposure or reuse that could lead to serious illness or even death. Past disposal techniques involve the requirement of medical facilities to cut the needle from the syringe body immediately after injection. This procedure, however, has been discovered to spread disease through airborne aerosols caused by the mechanical sheering action. The contaminated needle tip and syringe then still need to be handled and disposed of as a regulated waste item. More recent developments have led to depositing the syringe and needle into a “sharps” container. The sharps container would then be delivered to an authorized facility in a costly tracking, treatment, and disposal process.

Existing methods and systems, such as incineration, autoclaving, chemical treatment, electronic beam radiation, gamma rays, microwave energy, use of a low-voltage electric current (to destroy a needle at the point of use), encasing needles in resins or gels, and the like, have numerous shortcomings. Those shortcomings include inefficiency, high cost, high possibility for human error, inability to handle both sharps plus red-bag medical waste, and creation of infectious and hazardous fumes when the medical waste is treated. In an effort to address some of these shortcomings, methods and systems employing a heat treatment process have been reported in U.S. Pat. No. 5,972,291 and U.S. 2007/0224077 and are hereby incorporated in their entirety. These systems have shortcomings in ease of use, flexibility, efficiency, and automation. Furthermore, there remains a need for more rapid cycle times.

### SUMMARY OF THE INVENTION

The present invention is directed to a non-incinerating medical waste treatment device of limited volume displacement (e.g., “footprint”) and methods of use. The device contains two to four thermal chambers with total independent operation and controls. The desktop mountable device contains electronic, electromechanical, and made-up mechanical assemblies. The intended application of the device and method is for neutralizing medical waste such as biological contaminated bandages and/or sharps in order to eliminate and reduce biological contamination to disposal sites.

The device is a fully operational self-contained temperature- and time-controlled thermal oven with electro-mechanical lockout. The device and method are controlled by embedded microcontroller(s) operated by custom software/firmware and various electro-mechanical mechanisms. Additionally, the device is equipped with performance monitors and operational deliberate lockouts.

### BRIEF DESCRIPTION OF THE FIGURES

FIG. 1 shows a perspective front view of the device with the top closure doors for the two chambers in the closed position.

FIG. 2 shows a perspective front view of the device with one of the top closure doors for the two chambers in the open position.

FIG. 3 shows a detailed view of a closure door in the open position displaying the top portion of the heating chamber.

FIG. 4 shows a more detailed view of latching mechanism for the closure door to the heating chamber.

FIG. 5A shows a view of the heating chamber in a chassis enclosure with heating plates, thermocouples, and cooling fins.

FIG. 5B shows the same view of heating chamber as in FIG. 5A, with the addition of insulated padding with reflective Mylar film on the outer metal shell to aid in thermal isolation.

FIG. 6 shows a cutaway view of the two chambers and their exhaust tubing, UV system, and gas filtration system.

FIG. 7 shows a view of the UV system with a UV light source.

FIG. 8 is a schematic representation of the central controller in the device and how it controls the device through a touchscreen display to regulate the thermal cycling of medical waste.

FIG. 9 shows examples of Pulse Width Modulation cycles that can be used in the thermal cycling of both chambers.

FIG. 10 shows a representation of the product following the thermal treatment.

### DETAILED DESCRIPTION OF THE INVENTION

### Definitions

Unless specifically indicated otherwise, all technical and scientific terms used herein have the same meaning as commonly understood by those of ordinary skill in the art to which this invention belongs. The following terms shall have the meanings stated therewith.

Device having the meaning of an apparatus, instrument, system, or machine.

Medical waste, biological waste, biohazardous medical waste, or infectious waste includes contaminated needles, scalpels, sharp metal, glass objects, and other objects such as soft wastes items including gauze, tape, fabrics, and the like, that come into contact with the human body or bodily fluids. Medical waste includes waste generated in hospitals, medical clinics, doctor's offices, dentist's offices, blood banks, blood collection sites, and the like.

Container, cannister, or bin refers to a receptacle capable of receiving medical waste for processing by the device.

Chamber is a cavity capable of heat processing of a container or cannister of medical waste.

Throughout the description and claims of this specification, the word “comprise,” (and variations of the word, such as “comprising” and “comprises,”) means “including but not limited to,” and is not intended to exclude, for example, other additives, components, integers, or steps.

The phrase “and/or” as used herein, should be understood to mean “either or both” of the elements so conjoined, i.e., elements that are conjunctively present in some cases and disjunctively present in other cases.

As used herein, “or” should be understood to have the same meaning as defined above for “and/or”. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of”, or, when used in the claims, “consisting of”, will refer to the inclusion of exactly one element of a list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of.”

The device described and detailed herein is a self-contained fully automated non-incinerating medical waste treatment device of limited volume displacement (e.g., “footprint”). The device can be placed on a countertop or a mobile-care mountable entity (e.g., cart). Certain exemplary embodiments of this invention comprise medical waste treatment devices capable of processing infectious medical waste (red bag medical waste), except human and animal body parts, radioactive waste, and chemotherapeutic waste (depending on state regulations). In general, medical waste is comprised of different types of components such as plastic, cotton, aluminum, glass, etc. Generally, “sharps” material refers to needles, syringes, IV tubing, and the like, while soft or “red-bag” waste refers to gauze, cotton balls, tubing, gowns, etc., which may be contaminated with potentially infectious fluids such as blood and the like.

The device has a front compartment and a back compartment. The front compartment is comprised of an anti-microbial plastic enclosure having a user interface, central controller, and removable liquid waste collection receptacles. The back compartment or chamber compartment contains from two to four chambers, wherein each chamber is capable of receiving a container or container of medical waste ranging in volume from about 1 pint to about 4 gallons. Each chamber is fitted with a closure door or lid and a locking mechanism to enclose or seal the medical waste container during the thermal processing of the medical waste. In some embodiments, the chambers are thermally isolated and electrically independent. Thermal processing for each chamber is independently controlled and can operate either sequentially or simultaneously.

The device has active temperature monitoring by means of fixed temperature thermo-couplers and silicon-type sensors; redundant circuitry and sensors are required. There are thermal sensors mounted directly to the aluminum chamber housing and one or more silicon sensors set to measure air temperature within each of the chambers. The device may incorporate thermal active switches set to upper-limit specification mounted through and mechanically affixed to each chamber. The device is capable of maximum temperature of 232° C. (450° F.) at a tolerance of +/−2° C. (+/−4° F.). The thermal cycle temperatures may be established and locked into the device.

In some embodiments, the chambers are fitted with at least one plate heater coupled to an exterior surface of the chamber for providing heat to the container and a plurality of fins formed on an exterior surface of at least one side of the chamber for cooling the chamber. The chamber is built of extruded aluminum and assembled using a Hi-Temp (RED) RTV sealant (500° F. rated) applied to external and internal mating surfaces. In one embodiment, the Hi-Temp sealant is an epoxy adhesive.

In some embodiments, the chambers are heated using induction heat technology in air cooled induction coil assemblies. The induction coil chamber assembly can be in the form of a solenoidal coil enclosed in an epoxy potting material capable of receiving a medical waste container. The induction coil chamber provides for more rapid and uniform heating and cooling of the medical waste container within the chamber and thereby improving the device performance in processing and sterilizing the medical waste to a form safe for disposal.

In certain embodiments, the chambers are shaped such that a plurality of interior surfaces of each chamber contact a plurality of exterior surfaces of a container of medical waste when the container is received within the chamber. The container for receiving medical waste may comprise a container with a plug sealing the top end of the container. In one such embodiment, the medical waste container may be fabricated from a thermoplastic material such that exposure to dry heat will result in the container melting to encapsulate its contents. In another embodiment, the medical waste container is fabricated from a thermoplastic material and a non-removable lid such that exposure to dry heat does not melt the container and lid. After heat treatment, the sterilized container and lid is removed for safe disposal. Containers may be specific for receiving only red bag medical waste or sharps medical waste.

In some embodiments, a sleeve may be included to hold the container, and both the sleeve and container may be receivable within the chamber. The sleeve may be reusable and coated with a non-stick material on its interior and will fully contain the melted waste container after processing. In another embodiment, a container, rim, and plug are made of materials that withstand the thermal or dry heat cycle applied using such a system, in a way that they do not melt, while a chute and member in the container are made of a plastic material that melts and encapsulates the waste material (see U.S. 2007/0224077 A1).

In certain embodiments, the rim includes a plurality of slots spaced around a periphery of an inner portion of the rim, and the plug has a plurality of hooks that are received within the plurality of slots of the rim when the plug is engaged with the rim. The plug also may include a plurality of tabs spaced around a periphery of the plug. These backfill apertures may be found just above the projections of the rim, when the rim, container, and plug are engaged, to ensure that engagement of the rim and container is secure.

In another embodiment, a device for collection of infectious and medical waste comprises a container with a top-end opening for receiving waste; a rim configured to fit over an edge of and to engage the top end of the container; a chute configured to engage an inner portion of the rim, the chute extending into an interior of the container when engaged with the rim on the container; a member movably mounted within the chute and configured to limit access to a portion of the interior of the container beneath the member; and a plug configured to engage the rim such that the plug seals the opening of the container. This embodiment is particularly useful in the handling of sharps material. In another embodiment, the chute and the member are made of a transparent or partially transparent thermoplastic material.

In certain embodiments, the chute includes a flange that sits upon an inner portion of the rim when the chute and the rim are engaged, and the flange may have a plurality of notches spaced around it and positioned to avoid interfering with engagement of the rim and the plug. In a preferred embodiment for use in a heat treatment system, the chute and member are made of a plastic material that melts and encapsulates the waste material, while the container, rim, and plug are made of materials that withstand the dry heat cycle applied using such a system. The member may be mounted on posts of the chute and include a plurality of fins defining areas that receive the waste. Each fin is configured to rotate and dump waste into the portion of the interior of the container beneath the member when waste is placed into one of the areas between the fins.

In certain embodiments, the container is made using a thermoplastic material with a melting point at or below 340° F. In such embodiments, the rim may be integrated into the outer body of the container, including (as in other embodiments) mechanisms for attaching the chute and member. In such embodiments, for use in a heat treatment system, a plug is not used, and the container and rim, chute, and member are all made of a plastic material that melts and encapsulates the waste material within the container. When a container is used in this manner, it may be placed within a reusable sleeve within the chamber of a heating system, as mentioned above.

The chamber operates as a sealed assembly, and when cover assembly is closed and latched, there is only one exhaust gas venturi/port exiting each of the cavity chambers and this venturi/port is interconnected through a series of tubes and various mechanical connectors to the gas filtration system and liquids collection reservoir.

The device includes metal tubing (e.g., copper tubing) that allows venting of liquids and gases generated during the heat processing of the waste material for each chamber. Turning to the tubing for the first chamber, this tubing is attached to a single exhaust port (e.g., venture) located in the upper portion of the chamber. Shortly after leaving the chamber, the tubing is fitted with a thermal break that prevents the tubing from transmitting heat throughout its length. The tubing continues into a downward spiral section ending at a T-valve (e.g., Tee fitting). Attached to the T-valve are a second tube (e.g., copper) and third tube (e.g., copper). The second tube is configured to allow venting of gases and is connected to an ultraviolet (UV) system. The UV system comprises an aluminum enclosure containing a 185 nm bulb (UV element) and two quartz tubes running parallel to the bulb through which the gasses pass. One of the quartz tubes is used for the gases from the first chamber, while the second quartz tubing is used independently for gases from the second chamber. The UV system allows one light source to process two independent gas streams. The third tube is configured to allow the condensed liquid to flow into a removal collection receptacle (e.g., serviceable reservoir tank). Similarly, the second chamber is fitted with tubing to allow for the separation and purification of generated waste gases and liquids. As described above, the tubing for the second chamber incorporates the same ultraviolet system. A fourth tube juxtaposed to the second tube is attached to charcoal filters from which the gases are allowed to vent out the back of the filtration system, where a venting fan exhausts the purified gases out to the atmosphere. In some embodiments, the charcoal filter(s) will have a screw-type mating through to a threaded cover. Optionally, the tubing used to separate the liquid waste can have a different UV filter system in-line prior to collection.

In some embodiments, the device may contain more than two chambers, in which each chamber is comprised of similar mechanical tubing to allow for venting of gases and liquids generated during the thermal processing of the waste material. The UV system may be expanded to include additional quartz tubing, or an additional UV system may be incorporated for the additional chambers.

The device is fitted with air circulation and ventilation systems to independently manage the cooling of the front and back compartments. The air circulation and ventilation system for the front compartment, containing the user interface, central processor, and removal liquid collection receptacles, may contain one or more air intake fan(s) and exhaust vent(s) that allow for directional air flow and circulation. In some embodiments, the one or more air intake(s) do not comprise a fan allowing for passive air flow, while the exhaust vents do contain fans that allow for directional air flow and circulation.

The air circulation and ventilation system for the back or chamber compartment comprises a plurality of air intake fans and exhaust vents that allow for directional air flow and circulation. In some embodiments, the air intake vents do not have fans while the exhaust vents do have fans.

The device incorporates, in the front compartment, one or more central microprocessor-based controller(s). The central controller is set to safely operate, monitor and control the entire operation of the device. The central controller operationally controls the electro-mechanical lockouts to prevent the accidental opening of the chambers while in a heat cycle mode of operation controlled by the central controller. The device is capable of maintaining the lockout of failed operational cycles in the event of power failure while the device is operating in the heat cycle mode. In some embodiments, the central controller is capable of controlling from two to four cavities. The central controller has built-in diagnostics to aid in troubleshooting various failure modes and components. Specifically, while in operational mode, the device is capable of active monitoring and of providing dynamic and operational data on demand. The central controller may have the capability of setting a temperature profile or using a standard embedded profile.

In some embodiments, the central controller has embedded radio transceivers, specifically Wi-Fi and Bluetooth communication transceivers, allowing for the remote gathering of data and diagnostics. The display section may contain an audible device.

The device incorporates a local user interface in the display located in the front compartment that allows for total control of features and functions of the device. Remote accessing of the device is limited to monitoring and data collection features and functions. The display section located in the front compartment operates over a wide temperature range, preferably about 10° C. to about 60° C. without display clearing/washout. There are two mechanical switches, one to the rear panel, (main power), and the rear panel switch is the power main switch and circuit breaker as required; the second switch is the input power selector VAC 110/220 located adjacent to the power switch and integrated to the power plug assembly. Provisions of Universal Serial Bus (USB) ports are included for hardware maintenance and retrieval of data and/or diagnostics and can be located in the front compartment and rear device panels. The rear device panel USB serves as a dedicated local printer port in the event of Bluetooth and Wi-Fi loss.

In certain embodiments, a typical process cycle time for a container of medical waste is approximately one and half hours to two and one-half hours. The system takes approximately 10 to 20 minutes, depending on mass of the container, to heat from room temperature to a temperature in the range of about 300° F. to about 425° F., more preferably 350° F. to 400° F., after the container is placed in the heat chamber of the device. The use of induction heating will shorten the time to reach the operation temperature to between 2 to 5 minutes. After the chamber reaches the desired temperature, the container of waste is held and heated at this temperature for about 30 to 120 minutes, preferably 60 to 90 minutes, and more preferably about 90 minutes. the system is maintained in the closed and locked position as the medical waste is heat-processed. The container is then allowed to cool to at safe handling temperature of approximately 120° F. or less before the container can be removed. This cool-down takes about 15 to 30 minutes. It should be understood that embodiments of systems and methods of this invention are not limited to the cycle times disclosed above, which are merely exemplary.

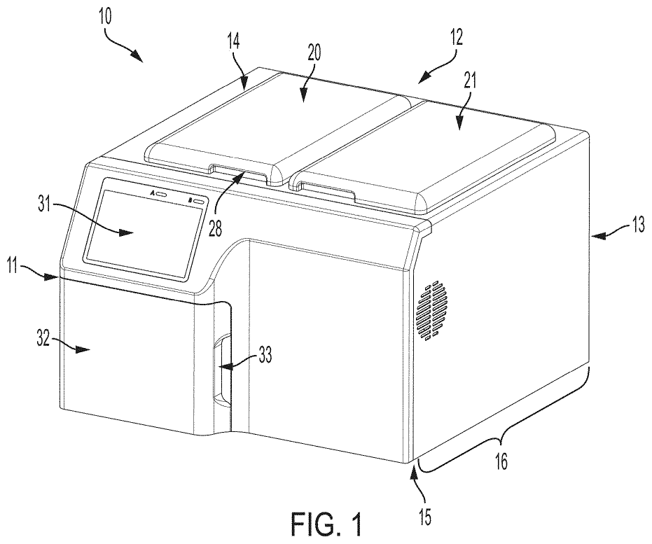

Referring now to the figures, an embodiment of a self-contained fully automated non-incinerating medical waste treatment device 10 of this invention is shown in FIG. 1. Device 10 includes a front portion 11; a back portion 12; a side 13; a top 14; a bottom 15. The device 10 being divided internally into a front compartment and a back compartment 16 (hidden by the device housing). Front portion 11 has a user interface 31 and a door 32 with handle 33 enclosing removable liquid collection receptacles (not shown). The front portion 11 is comprised of a heat resistant engineered antimicrobial thermoplastic. Side 13 has vent to allow directional air flow in the front portion 11. Closure device (lid or door) 20 and 21 are provided on the top of device 10.

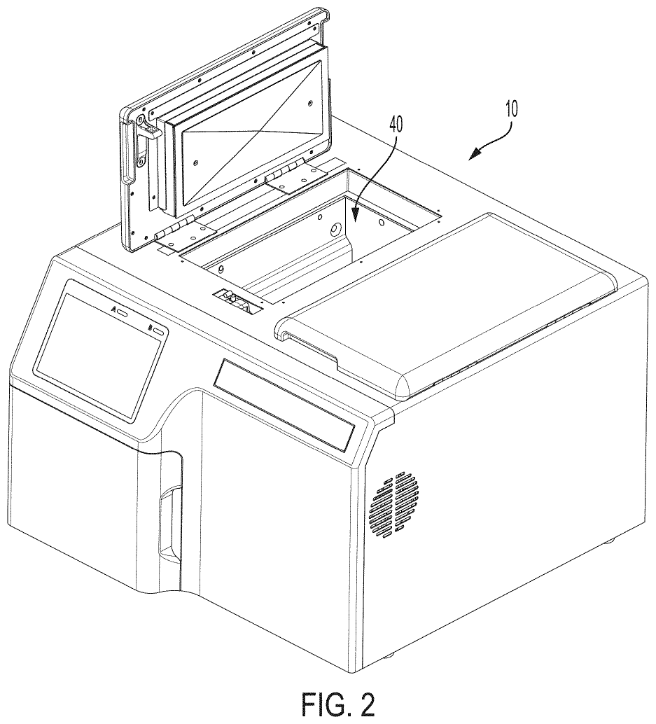

FIG. 2 shows device 10 with closure device 20 open to allow access to a heat chamber 40. Similarly closure 21, in the closed position, opens to allow access to a second heat chamber 41 (not shown). Containers of medical waste (not shown) are placed into the chambers 40 (shown) and 41 (not shown) and treated as further described below.

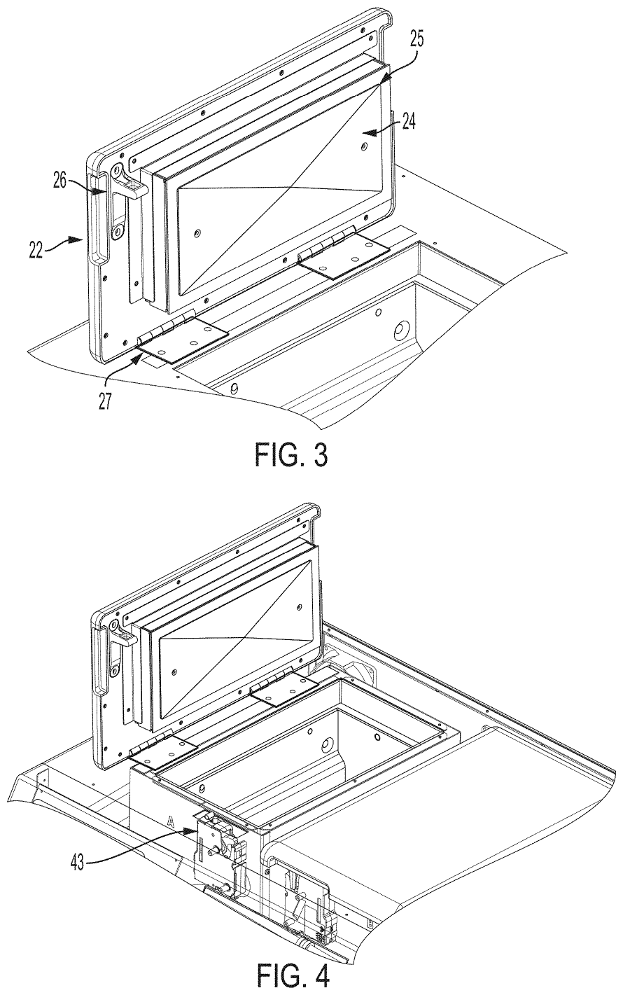

FIGS. 3 and 4 show the closure device 20 in FIG. 3 (likewise closure device 21 in FIG. 4) having an upper portion 22 formed of a heat resistant engineered antimicrobial thermoplastic and a lower portion 24 that together make up the door or lid of the closure device. The lower portion has a seal 25 that forms an airtight seal when the closure device is in the closed position. The closure device 20 has a latch 26 affixed to it that forms one part of the latching system (see FIG. 4, 43). Latch 26 may be engaged with receptacle 43 to secure closure device 20 in the closed position, such as, for example, during operation of device 10. Mounting brackets 27 secure closure device 20 to the top of the body of device 10. Mounting brackets 27 are pivotal, spring-like, and/or hydraulic in nature to allow closure device 20 to be moved from open to closed positions. Other structures, such as sliding devices, caps, screw-on lids, etc., could be used as well to control access to chamber 40 (FIG. 2). A handle 28 (FIG. 1) is also present on closure device 20. The back compartment 16 (FIG. 1) is encompassed by any rigid material capable of withstanding the heat ranges and cycle times described herein, such as stainless-steel sheet metal or heat resistant thermoplastics. In one embodiment, the material used is galvanized annealed steel.

In one embodiment, device 10 (FIG. 1) has approximate dimensions of about 21 inches deep by about 20 inches wide by 15 inches high, but it should be understood that embodiments of devices of this invention may be of any suitable size.

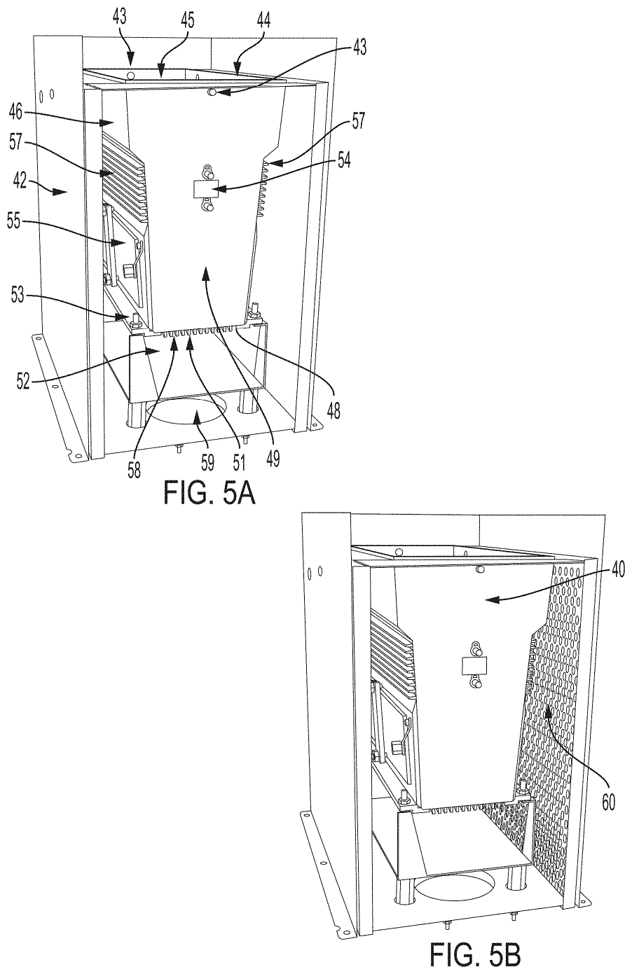

FIG. 5A shows a view of the chamber 40 (FIGS. 2 and 5B) (similarly for chamber 41, not shown) is shown within a body of device 10. A container, such as a container (not shown) is positioned within chamber 40 (FIG. 5B). The chamber 40 is formed of any suitable rigid material capable of withstanding the heat ranges and cycle times described herein. In one embodiment the chamber 40 is comprised of aluminum. In another embodiment, there are thermal sensors mounted directly to chambers 40 and 41 (not shown) and silicon sensor set (not shown) to measure air temperature within each of the chambers.

Chamber 40 (see FIG. 5B) includes a plurality of posts 43 spaced around the exterior periphery of chamber 40. Rim 44 surrounds the periphery of chamber 40 but does not cover an opening 45 at the top end of chamber 40 that receives a container of medical waste to be treated using device 10. Chamber 40 has long sides 46 and 47 (not shown), a bottom 48, and short sides 49 and 50 (not shown). In one embodiment, sides 46 and 47 (not shown) and bottom 48 are extruded aluminum, sides 49 and 50 (not shown) are cut or stamped pieces of aluminum, and sides 46 and 47 (not shown), bottom 48, and sides 49 and 50 (not shown) are welded together to form the central structure of chamber 40 (FIG. 5B). In another embodiment, the entire chamber body may be cut or stamped from sheet aluminum, folded into shape and the seams welded. Bottom 48 includes flanges 51 extending generally transversely out from bottom 48. In one embodiment, flanges 51 are formed during extrusion of bottom 48. Chamber 40 is secured to mounting bracket 52 with fasteners (not shown) that extend through holes (not shown) formed in flanges beneath nuts 53. In one embodiment, chamber 40 may be made in three pieces, with sides 46 and 47 (not shown) and bottom 48 extruded as a unshaped channel and sides 49 and 50 (not shown) fabricated and attached by welding or other means. In one embodiment, chambers 41 and 42 are encompassed within the body 42.

A thermal limiter 54 is mounted or coupled to side 49. Thermal limiter switches are well known to those skilled in the art, and thermal limiter 54 is provided to turn off plate heaters 55 and 56 (not shown) in the event chamber 40 becomes overheated. Thermocouples are disposed on sides 46 and 47 (not shown). Thermocouples measure the temperature of the exterior surfaces of those sides of chamber 40. Plate heaters 55 and 56 (not marked) are electrically connected to power through thermal limiter. In some embodiments, two 500 W heaters are utilized to achieve the upper temperature limit as required, one to each long side 46 and 47 (not marked) of chamber 40. Heaters 55 and 56 are fastened to the exterior of the chamber tub on long sides 46 and 47 and bolted in place, between the back of the flat-pack heater and the chamber wall, a silicon thermal gasket may be installed, the thickness of the gasket being about 0.25 mm. If the temperature of chamber 40 at the location of thermal limiter 54 exceeds a designed set point of thermal limiter 54, thermal limiter 54 will break the power circuit to plate heaters 55 and 56 (not marked), shutting them down to prevent overheating of device 10.

A plurality of fins 57 are formed on the exterior surface of sides 46 and 47 (not shown). Similarly, a plurality of fins 58 are formed on the exterior surface of the bottom 48. In one embodiment, the sets of fins 57 and 58 are formed in sides 46 and 47 (not shown) and bottom 48 during extrusion. In the folded embodiment of the chamber, extruded finned aluminum may be mechanically joined to the sides and/or bottom of the chamber. FIGS. 57 and 58 are incorporated into chamber 40 to assist in cooling the chamber rapidly after the completion of heat processing of a container of medical waste. Cooling air flows directionally through the system and exits the device through vent holes on the bottom portion 59.

FIG. 5B shows another embodiment of the chamber 40 (similarly for chamber 41) within a body 42 of device 10. Attached to the walls of the inner surface of body 42 are thermal insulated pads 60 with reflective Mylar film that aids in thermal isolation and overall thermal efficiency.

A container of waste, such as a container, is placed into chamber 40 (similarly for chamber 41) when closure device 20 (likewise for 21) is in the open position. The sides of container contact the interior surfaces of sides of chamber 40 in the area where plate heaters 55 and 56 (not shown) are coupled to chamber 40. In other embodiments, chamber 40 may be slightly larger than the container of waste that it receives such that a very minimal open space exists between the container and all interior surfaces of chamber 40. In other embodiments, a sleeve may be reusable and placed inside chamber 40, and a container designed to melt may be placed within sleeve, such that the sides of sleeve contact the interior surfaces of chamber 40.

Once the container is in position, closure device 20 is closed and latch 26 is engaged into receptacle 43 (see FIGS. 3 and 4). Solenoids may be used to power the latching and also power the unlatching. The device is outfitted with thermal lockout and has brown-out protection to prevent latches from opening after power loss and/or restoration.

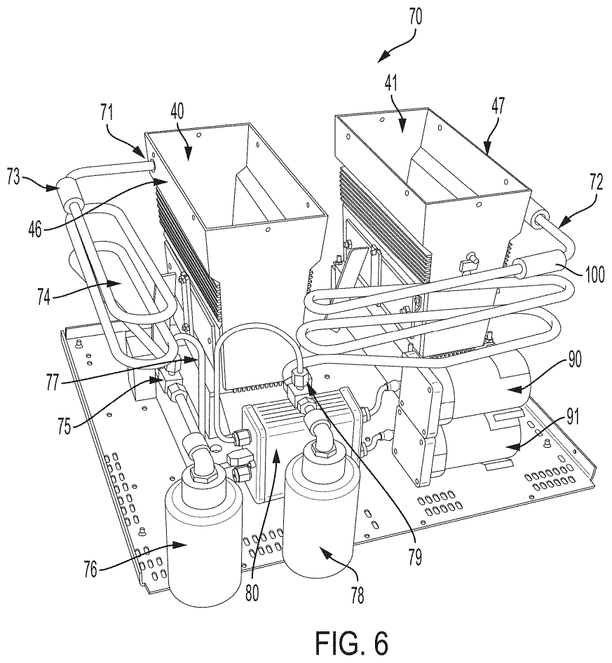

A lower portion 24 (FIG. 3) of closure device 20 (FIGS. 3 and 4) extends within body of the chamber 40 and effectively seals the top opening of chamber 40 (FIG. 5A). There is no airflow in or out of chamber 40, except through exhaust tube 71, as described above and described further below (see FIG. 6). As plate heaters 55 (FIG. 5A) and 56 (not shown) are brought to the proper temperatures for sterilization and/or to render the waste material unrecognizable and not reusable, a user of device 10 can monitor the container through the display and control panel 31 on front portion 11 of body of device 10.

It should be understood that when medical waste, or any waste comprised of different materials, is heated, there is typically no orderly heating of the material. The waste material does not undergo an even or uniform heating, as there are different compositions and localized volumes within the container being heated. For example, if a pocket of alcohol is heated within the medical waste, it will start outgassing before the surrounding materials because of the low vapor point of the alcohol. This will produce a rapid and voluminous flow of gas. Due to this random heating, device 10 is designed to handle any associated unpredictable heating and gas expansion problems that may occur.

The heat chamber exhaust flow, or exhaust flow, is shown in FIG. 6. Exhaust tubing 71 with an exhaust gas venture/port exit is secured to side 46 of chamber 40. Similarly exhaust tubing 72 with an exhaust gas venture/port exit is secured to side 47 of chamber 41. The exhaust tubing may be made of a metal, e.g., copper, that can withstand holding gases at high temperatures. The tubing 71 which carries the exhaust exiting from the chamber 40 through a thermal break 73 that prevents the tubing 71 from transmitting heat throughout its length. The exhaust continues down the tubing 71 through a spiral section 74 where it enters a Tee fitting 75, where the liquid component of the exhaust continues to the collection vessel 76 through a second metal tubing, and the gaseous component is directed through a third metal tubing 77 to a UV system 80 and passes out of the UV system into a filtration system 90 followed by venting out the back of the filtration system where the purified and decontaminated gas is vented outside the device. In some embodiments, filtration system 90 is comprised of charcoal filter(s) which may have a screw-type mating through to a threaded cover. Similarly, the exhaust tubing 72 for chamber 41 performs the process of cooling and separating the liquid from the gaseous component of the exhaust. The tubing 72 has a thermal break 100, a spiral section 101, and Tee fitting 79 where the liquid and gaseous components of the exhaust are separated. The gaseous component passes through metal tubing to the UV system 80 in-line with a second filtration system 91. Likewise, filtration 91 can be comprised of charcoal filter(s).

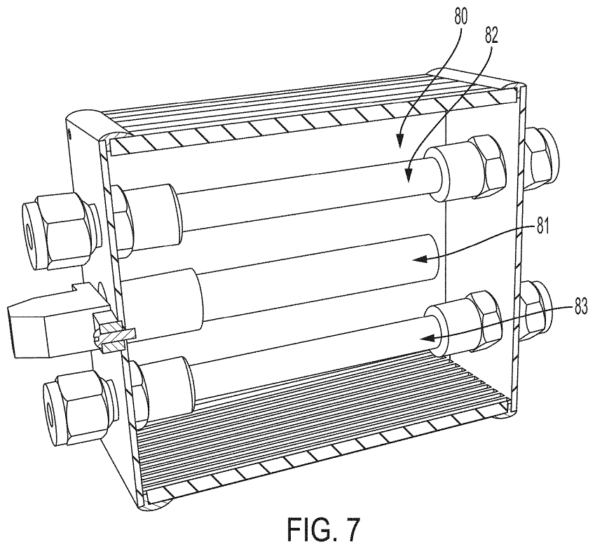

The UV system 80 is shown in more detail in FIG. 7. The UV system 80 may have an aluminum enclosure containing a 185 nm bulb (UV element) 81 and two quartz tubings running parallel to the bulb 82 and 83 in which the gasses pass. One of the quartz tubing is used for the gases from the first chamber 40 while the second quartz tubing is used independently for gases from the second chamber 41. The UV system is thus capable of allowing a single UV light source to process two independent gas streams.

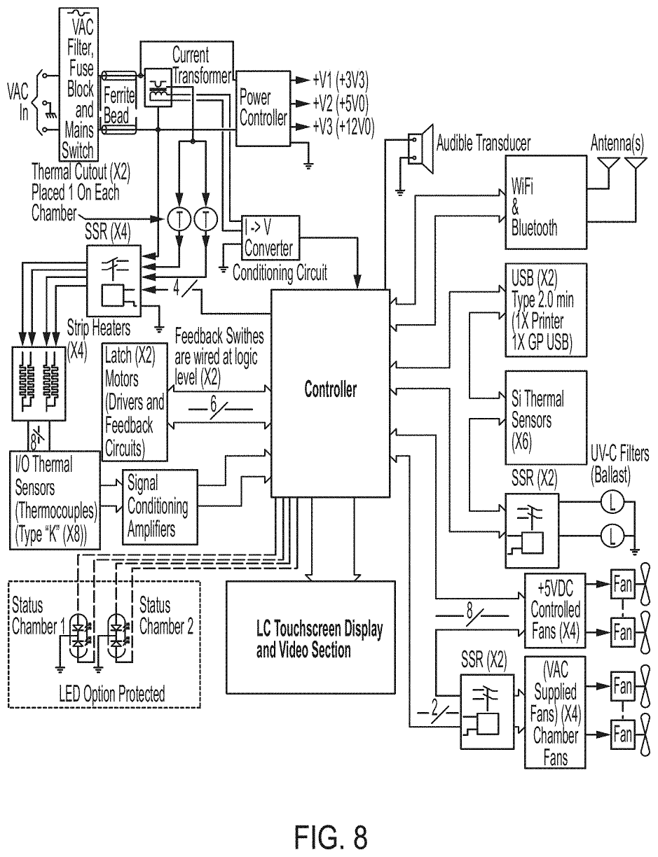

FIG. 8 shows a schematic representation of the central controller. The central controller is set to operate, monitor, and control the entire safe operation of the device.

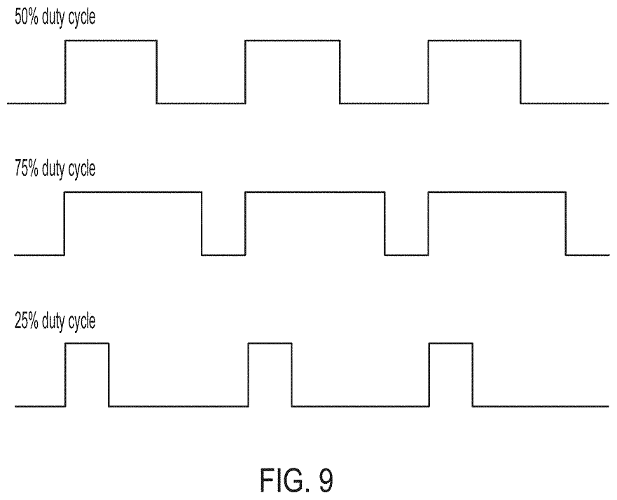

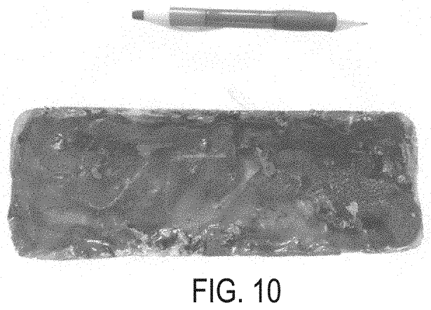

The heaters 55 and 56 for heat chambers 40 and 41 consume each about 1,000 watts of power. For the system to operate using a standard 110V wall outlet, total power must be managed. To manage the power levels of the device, Pulse Width Modulation (PWM) may be employed to control the heating cycles of chambers 40 and 41 (see FIG. 9).

Chamber 40 receives 100% power to reach steady state, then receives periodic power input to maintain temperature with a low duty cycle if and when the second chamber 41 cycle is activated. Examples of 50%, 75%, and 25% low duty cycles are shown in FIG. 9.

Chamber 41 when Chamber 40 is at steady state receives higher duty cycle to ramp up (at a slower rate) to steady state. PWM signals for chambers 40 and 41 are always out of phase of each other. When both chambers are at steady state, power cycles to one or the other based on feedback from individual chamber temperature sensors.

### EXAMPLES

### Example 1

### Method of Use of the Medical Waste Device

The self-contained fully automated non-incinerating medical waste treatment device comprising two chambers is conveniently located on a countertop or cart in a room or space for the safe disposal of generated medical waste (e.g., sharps or red-bag waste). The device is capable of processing up to 10 gallons of medical waste in a day using both chambers (double batch).

Containers for receipt of the medical waste are placed in each of the receiving chambers of the device.

Containers may be specific for containing only red bag medical waste or sharp medical waste. The containers receive the medical waste outside of the medical waste device.

The user activates the device by a simple key entry sequence.

The activated device initiates a process in which the closure lid to the chamber containing the container is latched and locked down and a heating cycle is initiated in the chamber to heat the chamber to a predetermined target temperature, preferably 350° F. to 400° F. The device can be fitted with a temperature sensor to measure the temperature of the contents of the container. The target temperature is maintained for a set time, preferably about 60 to about 90 minutes, after which the heater for the chamber is automatically turned off, a cooling fan may be activated, and the device cools the container to a temperature appropriate for safe handling. The lid is kept locked until the temperature is below 118° F. (48° C.). An LED indicator light remains lit while the chamber temperature is above this temperature. An example of a processed container is shown in FIG. 10.

During the operation of the device the external surfaces of the device remain cool. The device is noise-free during operation.

Following the completion of the process, labels are generated for incorporation into a compliance binder for adherence to the processed container(s). The labels can contain processing data, including data on the treatment cycle allowing for automatic documentation for compliance and regulatory requirements. The processing data is stored in the device and can be uploaded to a cloud service for maintaining a permanent record.

The device is capable of processing both containers independently.

The device is capable of processing both containers simultaneously or sequentially.

The system has redundant safety features ensuring that the temperature is properly maintained to meet all treatment and emission standards.

The system is equipped with a filtration system to prevent odors and act as a redundant safety measure to make certain all emissions are bacteria-free. A series of redundant fail-safe software and hardware features have been incorporated to ensure that all waste is decontaminated, with the sharps and medical waste having been rendered sterilized and safe for handling and disposal.

If the process conditions have not been met for any reason, the system remains locked down and reports the failure to Technical Support for diagnostics. The operator will not be allowed to access the collector until the problem has been resolved.

On a predetermined basis, each device will automatically contact the Quality Systems Database to upload process data on each treatment cycle. Quality Systems will analyze an extensive set of data to check that all systems and components are operating within recommended tolerances. An e-mail can be automatically generated that summarizes process data for each treatment cycle, as a redundant compliance reporting service.

### Example 2

### Medical Waste Device Sterilization Test

Medical waste collection containers were configured to separately evaluate a sharp biohazardous load and a red bag biohazardous waste load. These loads were contaminated with a battery of microorganisms, processed through the device decontamination cycles, and evaluated for efficacy. Water emissions from the loads were also evaluated.

The following tests for the efficacy of the medical waste device follow concepts, requirements, and guidance outlined in ISO 11138-1, ISO 11138-3, ISO 11138-4, ISO 20857, ISO 17665-1, and ISO 14937.

Sharps Load Configuration: Approximately 370 g of syringes (multiple sizes) with cannulas attached and a small quantity of water (25-50 mL).

Red Bag Load Configuration: Approximately 170 g of absorbent material moistened with a mixture of 50-100 mL of water and 5 mL of defibrinated blood test soil.

Test Organisms: Staphylococcus aureus, Pseudomonas aeruginosa, Candida albicans, Aspergillus brasiliensis, Mycobacterium terrae, Mycobacterium hassiacum, Bacillus atrophaeus, and Geobacillus stearothermophilus.

### Test Method Acceptance Criteria

Ampoule Testing: Ampoules must have a minimum population of 1.0×10⁶ CFU (colony forming units) as determined from the positive control results. Additionally, the ampoule population should allow for a minimum 6 log₁₀ reduction to be measured.

Spore Strip Testing: Spore strips must have a minimum manufacturer labeled population of 1.0×10⁶ CFU/strip as listed on the certificate of analysis (COA). Bls must have the labeled resistances (i.e. D values) and temperature coefficients (i.e. Z values) specified in the test protocol. Spore strips must show a BI population verification result of 50% to 300% of the manufacturer labeled population prior to use in testing.

Spore Suspension Testing: The G. stearothermophilus suspension must have a minimum labeled titer of 1.0×10⁸ CFU/mL, minimum D₁₂₁ value of 1.5 minutes, and a minimum Z value of 6° C. as labeled by the manufacturer. The spore suspension must pass BI population verification testing performed for received spore suspensions prior to use in testing.

Water Emissions Testing: Bacteriostasis testing will show the ability of the media to recover growth of <100 CFU of the indicator organism.

### Procedure

Biological Indicator (BI) Population Verification: A population verification was performed on the spore strips and spore suspension utilized in testing (see table 1).

TABLE 1
Biological indicator population verification results

| | | | | | | | |
|---|---|---|---|---|---|---|---|
| | | | | | D- | | |
| | | | Labeled | Percent of | Value¹ | Z | |
| | | | Population | Manufacturer's | ~160° C. | Value² | Expiration |
| BI Type | Manufacturer | Lot # | (CFU) | Population | (min) | (° C.) | Date |

B. atrophaeus

| | | | | | | | |
|---|---|---|---|---|---|---|---|
| Spore Strip | Mesa Labs | BATR- | 2.1 × 10⁶ | 111 | 1.3 | 50.0 | 11 Nov. |
| | | 465 | | | | | 2021 |

G. stearothermophilus

| | | | | | | | |
|---|---|---|---|---|---|---|---|
| Spore Strip | Crosstex ® | RU91 | 1.7 × 10⁶ | 138 | 1.6 | 7.8 | 31 Mar. |
| | | | | | | | 2021 |

G. stearothermophilus

| | | | | | | | |
|---|---|---|---|---|---|---|---|
| Liquid | Crosstex ® | AR612 | 2.6 × 10⁸ | 96 | 2.0 | 7.7 | 30 Jun. |
| Spore | | | | | | | 2021 |
| Suspension | | | | | | | |

¹D-value or decimal reduction time is the time required, at a given temperature, to achieve a log reduction, that is, to kill 90% (or 1 log) of microorganisms.
²Z-value is the number of degrees the temperature has to be increased to achieve a ten-fold (1 log₁₀) reduction in the D-value.

Test Load Inoculation: Collection containers for sharps and red bag were loaded with contents specified above. The loads were then inoculated with appropriate organisms outlined in the table 2. The biological indicates (BI) and ampoules were placed in the upper middle portion of the load, which is furthest away from the heating element and is expected to be the most resistant to sterilization. The spore suspension was added directly to the load contents.

TABLE 2
Organisms used in sterilization cycle evaluation

| BI Type | Organism | ATCC #¹ | Organism Justification |
|---|---|---|---|
| Spore | B. atrophaeus | 9372 | Dry heat sterilization |
| Strip | | | indicator organism- |
| | | | spore former |
| Spore | G. | 7953 | Moist heat sterilization |
| Strip | stearothermophilus | | indicator organism- |
| | | | spore former |
| Ampoule | S. aureus | 6538 | Gram positive vegetative |
| | | | bacteria-clinically relevant |
| Ampoule | P. aeruginosa | 15442 | Gram negative vegetative |
| | | | bacteria-clinically relevant |
| Ampoule | C. albicans | 10231 | Yeast-Clinically relevant |
| Ampoule | A. brasiliensis | 164040 | Mold-Clinically relevant |
| | | | environmental |
| | | | contaminant |
| Ampoule | M. terrae | 15755 | High level disinfection |
| | | | indicator organism- |
| | | | clinically relevant |
| Ampoule | M. hassiacum | 700660 | Thermally-resistant |
| | | | disinfection indicator |
| | | | organism-clinically relevant |
| Liquid | G. | 7953 | Moist heat sterilization |
| Spore | stearothermophilus | | indicator organism- |
| Suspension | | | spore former |

¹American Type Culture Collection Number

Sterilization Cycle Evaluation: Each test load was prepared as listed above. The prepared collector containers were placed into the medical waste device. The decontamination cycles were run at a temperature set point of 350° F.

Following the completion of each cycle, the test load was removed from the medical waste device. The Bis/ampoules were removed from the load and transferred to a HEPA-filtered hood for analysis. The water emissions containers were also transferred to a HEPA-filtered hood for analysis.

The Bis were transferred to sterile containers filled with soybean casein digest broth (SCDB). The G. stearothermophilus Bis were incubated at 55-60° C. for great than or equal to 7 days and observed for growth of the challenge organism. The B atrophaeus B is were incubated at 30-35° C. for great than or equal to 7 days and observed for growth of the challenge organism.

The ampoules were tested by filtering 1.0 mL of the organism suspension and plating the filter onto an appropriate type of agar. Positive control ampoules were tested by performing a standard plate count on each vial. Dilutions were tested as necessary. The S. aureus samples were plated on MSAG and incubated at 30-35° C. for 1 to 6 days or until growth of the positive was apparent. The P. aeruginosa samples were plated on CEAG and incubated at 35-39° C. for 1 to 6 days or until growth of the positive was apparent. The C. Albicans samples were plated on PDXA and incubated at 20-25° C. for 1 to 6 days or until growth of the positive was apparent. The A. brasiliensis samples were plated on SDEX and incubated at 20-25° C. for 3 to 5 days or until growth of the positive was apparent or the colonies were easily counted. The M. terrae samples were plated on 7H11 and incubated at 35-39° C. for 14 to 21 days or until growth of the positive was apparent or the colonies were easily counted. The M. hassiacum samples were plated on 7H10 and incubated at 35-39° C. for 7 to 14 days or until growth of the positive was apparent or the colonies were easily counted.

The water emissions samples were test by filtering the entire volume of liquid recovered from the cycle and plating the filter onto CTSA and incubated at 55-60° C. for 24 to 48 hours or until growth of the positive was apparent.

Bacteriostasis Testing (for water emissions): If a device being tested for sterility contains bacteriostatic substances, no microorganisms will grow, even if they are present. This procedure verifies that if a negative sterility result is produced, it is due to the absence of microorganisms and not due to inhibitory substances inherent to the materials tested.

Cycles were run in the medical waste device using each load configuration as outlined previously. These loads were run without including any of the test organisms. The emissions from the loads were collected after completion of the cycles. The emissions were then inoculated with <100 CFU of a G. stearothermophilus suspension. Membrane filtration testing was performed on the inoculated emissions samples and the filters were plated onto CTSA and incubated at 55-60° C. for 24 to 48 hours or until growth of the positive was apparent. Positive controls were performed by inoculating an appropriate volume of sterile water with the same amount of G. stearothermophilus as the emissions samples. Membrane filtration testing was performed on the positive control samples and the filters were plated onto CTSA and incubated at 55-60° C. for 24 to 48 hours or until growth was apparent. Recovery of the indicator organism was evaluated, and results of the emissions samples were compared to the positive control results.

### Results

TABLE 3
Efficacy Testing (Biological Indicators):

| Identification | | Run #1 | Run #2 | Run #3 |
|---|---|---|---|---|
| Sharps Load: | BI¹ #1 | 0 | 0 | 0 |
| G. stearothermophilus | BI #2 | 0 | 0 | 0 |
| | BI #3 | 0 | 0 | 0 |
| Red Bag Load: | BI #1 | 0 | 0 | 0 |
| G. stearothermophilus | BI #2 | 0 | 0 | 0 |
| | BI #3 | 0 | 0 | 0 |
| Sharps Load: | BI #1 | 0 | 0 | 0 |
| B. atrophaeus | BI #2 | 0 | 0 | 0 |
| | BI #3 | 0 | 0 | 0 |
| Red Bag Load: | BI #1 | 0 | 0 | 0 |
| B atrophaeus | BI #2 | 0 | 0 | 0 |
| | BI #3 | 0 | 0 | 0 |

| | | | |
|---|---|---|---|
| Positive Controls | +, +, + | +, +, + | +, +, + |
| Environmental Monitor | 0, 0, 0 | 0, 0, 0 | 0, 0, 0 |

0 = No Growth
+ = Growth
¹Biological Indicator

TABLE 4
Efficacy Testing (Organism ampoules)

| | | Run #1 | Run #2 | Run #3 |
|---|---|---|---|---|
| | | Log₁₀ | Log₁₀ | Log10 |
| Identification | Organism | Reduction | Reduction | Reduction |
| Sharps Load | S. aureus | >7.9 | >8.3 | >8.5 |
| | P. aeruginosa | >9.0 | >8.5 | >9.1 |
| | C. albicans | >6.8 | >7.1 | >6.7 |
| | A. brasiliensis | >8.0 | >8.0 | >7.9 |
| | M. Terra | >9.0 | >9.1 | >9.1 |
| | M. hassiacum | >7.4 | >7.4 | >6.5 |
| Red Bag Load | S. aureus | >7.9 | >8.3 | >7.7 |
| | P. aeruginosa | >9.0 | >8.5 | >9.1 |
| | C. albicans | >6.8 | >7.1 | >6.7 |
| | A. brasiliensis | >8.0 | >8.0 | >7.9 |
| | M. Terra | >9.0 | >9.1 | >9.1 |
| | M. hassiacum | >7.4 | >7.4 | >6.5 |

TABLE 5
Efficacy Testing (Water Emissions)

| | | Run #1 | Run #2 | Run #3 |
|---|---|---|---|---|
| | | Log₁₀ | Log₁₀ | Log10 |
| | | Reduc- | Reduc- | Reduc- |
| Identification | Organism | tion | tion | tion |
| Sharps Load | G. stearothermophilus | >6.4 | >6.7 | >6.7 |
| Red Bag Load | G. stearothermophilus | >6.4 | >6.7 | >6.7 |

TABLE 6
Efficacy Testing (Controls)

| | | Positive Control (CFU/mL) | | |
|---|---|---|---|---|
| Identification | Organism | Run #1 | Run #2 | Run #3 |
| Sharps Load | S. aureus | 8.0 × 10⁷ | 1.8 × 10⁸ | 3.5 × |
| Positive Control | | | | 10⁸ |
| | P. aeruginosa | 9.8 × 10⁸ | 3.2 × 10⁸ | 1.3 × |
| | | | | 10⁹ |
| | C. albicans | 6.7 × 10⁶ | ~1.4 × 10⁷ | ~5.6 × |
| | | | | 10⁶ |
| | A. brasiliensis | 9.4 × 10⁷ | 9.4 × 10⁷ | 8.4 × |
| | | | | 10⁷ |
| | M. Terra | 1.1 × 10⁹ | 1.3 × 10⁹ | 1.4 × |
| | | | | 10⁹ |
| | M. hassiacum | 2.7 × 10⁷ | 2.6 × 10⁷ | ~3.3 × |
| | | | | 10⁶ |
| | G. stearothermophilus | 2.3 × 10⁶ | 5.4 × 10⁶ | 5.1 × |
| | | | | 10⁶ |
| Red Bag Load | S. aureus | 8.0 × 10⁷ | 1.8 × 10⁸ | 4.9 × |
| Positive Control | | | | 10⁷ |
| | P. aeruginosa | 9.8 × 10⁸ | 3.2 × 10⁸ | 1.3 × |
| | | | | 10⁹ |
| | C. albicans | 6.7 × 10⁶ | ~1.4 × 10⁷ | ~5.6 × |
| | | | | 10⁶ |
| | A. brasiliensis | 9.4 × 10⁷ | 9.4 × 10⁷ | 8.4 × |
| | | | | 10⁷ |
| | M. Terra | 1.1 × 10⁹ | 1.3 × 10⁹ | 1.4 × |
| | | | | 10⁹ |
| | M. hassiacum | 2.7 × 10⁷ | 2.6 × 10⁷ | ~3.3 × |
| | | | | 10⁶ |
| | G. stearothermophilus | 2.3 × 10⁶ | 5.4 × 10⁶ | 5.1 × |
| | | | | 10⁶ |

TABLE 7
Efficacy Testing (Controls)

| | | Environmental Controls (CFU) | | |
|---|---|---|---|---|
| Identification | Organism | Run #1 | Run #2 | Run #3 |
| Sharps Load: | S. aureus | 0 | 0 | 0 |
| Environmental | P. aeruginosa | 0 | 0 | 0 |
| Monitor | C. albicans | 0 | 0 | 0 |
| | A. brasiliensis | 0 | 0 | 0 |
| | M. Terra | 0 | 0 | 0 |
| | M. hassiacum | 0 | 0 | 0 |
| Red Bag Load: | S. aureus | 0 | 0 | 0 |
| Environmental | P. aeruginosa | 0 | 0 | 0 |
| Monitor | C. albicans | 0 | 0 | 0 |
| | A. brasiliensis | 0 | 0 | 0 |
| | M. Terra | 0 | 0 | 0 |
| | M. hassiacum | 0 | 0 | 0 |

0 = No Growth
+ = Growth

TABLE 8
Bacteriostasis Testing (for water emissions)

| Exposure Phase Time | Article Identification | Results (CFU) |
|---|---|---|
| 30 Minutes | Sharps Load Replicate 1 | 23 |
| | Red Bag Load Replicate 1 | 45 |
| | Positive Control Replicate 1 | 38 |
| | Sharps Load Replicate 2 | 41 |
| | Red Bag Load Replicate 2 | 39 |
| | Positive Control Replicate 2 | 50 |
| 60 Minutes | Sharps Load Replicate 1 | 77 |
| | Red Bag Load Replicate 1 | 114 |
| | Positive Control Replicate 1 | 94 |
| 90 Minutes | Sharps Load Replicate 1 | 68 |
| | Red Bag Load Replicate 1 | 90 |
| | Positive Control Replicate 1 | 72 |
| | Sharps Load Replicate 2 | 9 |
| | Red Bag Load Replicate 2 | 6 |
| | Positive Control Replicate 2 | 9 |

TABLE 9
Attempted Cycle Set Points

| Cycle | Temperature | Exposure Time | Cycle | Load | Performance Criteria |
|---|---|---|---|---|---|
| Type | (° F.) | (minutes) | # | Configuration | Failures |
| Dry Heat | 350 | 30 | 1 | Sharps | G stearothermophilus |
| | | | | | BI |
| | | | | | G. stearothermophilus |
| | | | | | suspension (emissions) |
| Dry Heat | 350 | 30 | 1 | Red Bag | G. stearothermophilus |
| | | | | | BI |
| Dry Heat | 350 | 60 | 1 | Sharps | G. stearothermophilus |
| | | | | | suspension (emissions) |
| Dry Heat | 350 | 60 | 2 | Sharps | G. stearothermophilus |
| | | | | | suspension (emissions) |

### CONCLUSION

Table 3-9 show the effectiveness of the medical waste device with a temperature set point of 350° F. and exposure phase times of 30, 60, and 90 minutes to sterilize sharp load containers and red bag load containers containing various test bacteria. Also assessed as part of the effectiveness was the water emissions for levels of G. Stearothermophilus. Included in the effectiveness experiments were positive controls and environmental controls. Results showed the medical waste device met the effectiveness criteria for sterilizing sharp load configurations and red bag load configurations at a temperature set point of 350° F. and exposure phase time of 90 minutes as outlined in ISO 11138-1, ISO 11138-3, ISO 11138-4, ISO 20857, ISO 17665-1, and ISO 14937.

### Example 3

### Medical Waste Device Virucidal Test

The following virucidal effectiveness test of the medical waste device was conducted against the Human Corona Virus (H. Coy).

### Test Conditions

Number of cycles: 1
Test Temperature: 350° F.
Contact Time: 90-minute
Organic Load: 5% FBS (fetal bovine serum) in the inoculum
Organic Load: 10% FBS in medical waste

### Equipment and Reagents

Host cell growth medium: MEM with 10% FBS, lx PIS
Dilution medium: MEM with 5% FBS
Recovery host cell: MRC-5 ATCC CCL-171
Host cell incubation: 37±1° C., 5% CO₂, 90% RH (relative humidity)
Virus incubation in host cells: 34±1° C., 5% CO₂, 90% RH
Virus: Human Coronal Virus 229E, ATCC VR-740
96-wll plates
Biosafety cabinet
Humidified incubation
Microscope

### Method

### Cell Culture Preparation

Prepared multiple cultures of MRC-5 cells, in serum-supplemented Minimum Essential Medium Eagle, 1X.

Incubated cultures at 37±1° C. for not less than 24 hours in a 5±1% carbon dioxide atmosphere, until a monolayer, with greater that 80% confluence, is obtained.

Examine the prepared cultures under an inverted microscope to ensure uniform, near confluent monolayers

### Preparation of Challenge Virus

Cell lines were maintained in respective media thus the harvested viral stock contains 5% organic load.

Frozen viral stock was thawed on the day of the test.

An aliquot of 20 μL of virus stock was added to sterile metal carriers and the carriers allowed to dry completely under the biosafety cabinet at room temperature with the airflow on to facilitate drying.

Each cycle of test run has included 3 replicate carriers with virus and 1 carrier with medium only.

The drying time, relative humidity and temperature was recorded for each cycle test.

TABLE 10

| | Cycle I | Cycle II | Cycle III |
|---|---|---|---|
| Drying time | 27 min | 29 min | 25 min |
| Temperature | 24.9° C. | 23.5° C. | 22.8° C. |
| Humidity | 18.3% | 19.5% | 20.3% |

### Loading Medical Waste Device

Medical waste container filled to the maximum fill-line with simulated medical waste items including exam gloves, 3 cc syringes with needles, 3-ply cotton gauze pads, and cotton balls. The syringes with needle were ⅔ of the waste material.

The medical waste container was added with 50 mL MEM medium with 10% FBS (10% organic load).

The carriers located towards the collector's geometric center of the test device.

Carriers were kept in a glass petri plate placed an approximately 1-2″ below the maximum fill-line of the container.

### In Vitro Infectivity Assay for Recovery of Virus

Upon completion of the required cycles, the carriers removed and transferred to a fresh tube containing 2 mL of the dilution media and vortexed twice for 15 seconds each. Subsequent 10-fold serial dilution carried out with the dilution medium. Immediately then the dilutions inoculated into the host cells.

Host cells were plated in 96-well plate at suitable density 1 day prior to the test or monolayer reached to 70-80% confluency. Records of cell source, passage, seeding density, plate type and medium volume captured.

Aliquots of dilutions were inoculated into the well containing host cells, quadruplicate per dilution.

Inoculated plates were incubated at 34±1° C., 5% CO₂, RH 90%.

Presence or absence of viral infection was monitored for seven and recorded based on the viral cytopathic effect (CPE) on the host cells.

TABLE 11
Plate Conditions

| | |
|---|---|
| Cell Source | MRC-5 ATCC CCL-171 |
| Cell Passage | 35 |
| Seeding Density | 2 × 10⁵ cells/mL, 1 × 10⁵ cells/mL |
| Medium Volume | 100 μL/well |

### Controls

Cell Viability Control: The control demonstrates that indicator cells remain viable throughout the course of the assay period. In addition, it confirms the sterility of the cell culture system employed throughout the assay period. It also provides normal morphology in identifying CPE or cytotoxicity effect.

Virus Stock Control: Virus stock is tittered at the time of test to determine the relative infectivity of the virus. This control demonstrates that the employed indicator cell is susceptible to test virus infection.

Plate Recovery Control: In plate recovery control, the same volume of cell culture media is utilized to recover the dry virus, which keeps the initial viral load equivalent between plate recovery control and test group. The subsequent dilutions are performed same as the test material.

Cytotoxicity Control: The extent of cytotoxicity, if any, will determined for a proper range of 10-fold serial dilutions of the blank carrier processed at the same time of virus-loaded carriers. Cytotoxicity will be observed and recorded based on the morphological change of indicator cells.

Environmental Control: The relative humidity and temperature will be recorded during the test.

### Results

### Control and Results

Virus stock control demonstrate susceptibility to host cell line.

Virus recovery met the requirements with minimum 10⁴ TCID50 recovered.

Cell viability control met the requirements and no contamination occurred.

Cytotoxicity control met the requirements and no CPE induced by the test conditions.

TABLE 12
Human Corona Virus 229E (ATCC: VR-740)

| | Virus Control | | | | | | | |
|---|---|---|---|---|---|---|---|---|
| Dilution | (Recovered Virus) | | | | Virus Stock | | | |
| 10⁻² | + | + | + | + | + | + | + | + |
| 10⁻³ | + | + | + | + | + | + | + | + |
| 10⁻⁴ | + | + | + | 0 | + | + | + | + |
| 10⁻⁵ | + | 0 | 0 | 0 | + | 0 | 0 | 0 |
| 10⁻⁶ | 0 | 0 | 0 | 0 | 0 | 0 | 0 | 0 |
| 10⁻⁷ | 0 | 0 | 0 | 0 | 0 | 0 | 0 | 0 |
| TCID₅₀ (0.1 mL) | 4.5 | | | | 4.7 | | | |
| Cell Viability | 0 | 0 | 0 | 0 | 0 | 0 | 0 | 0 |

Note
+ = presence of virus, 0 = unaltered morphology T = cytotoxicity

TABLE 13
Medical Waste Device Test Cycle #1 Against H. CoV
Test Cycle #1

| | Replicate 1 | | | | Replicate 2 | | | | Replicate 3 | | | | Cytotoxicity | | | |
|---|---|---|---|---|---|---|---|---|---|---|---|---|---|---|---|---|
| Dilution | (Virus) | | | | (Virus) | | | | (Virus) | | | | Control | | | |
| 10⁻² | 0 | 0 | 0 | 0 | 0 | 0 | 0 | 0 | 0 | 0 | 0 | 0 | 0 | 0 | 0 | 0 |
| 10⁻³ | 0 | 0 | 0 | 0 | 0 | 0 | 0 | 0 | 0 | 0 | 0 | 0 | 0 | 0 | 0 | 0 |
| 10⁻⁴ | 0 | 0 | 0 | 0 | 0 | 0 | 0 | 0 | 0 | 0 | 0 | 0 | 0 | 0 | 0 | 0 |

| Log10 TCID₅₀ | ≤1.5 | ≤1.5 | ≤1.5 | N/A |
|---|---|---|---|---|
| Log10 TCLD₅₀ | N/A | | | ≤1.5 |
| Log | ≥3.0 | ≥3.0 | ≥3.0 | N/A |
| Reduction | | | | |

N/A = Not applicable

TABLE 14
Medical Waste Device Test Cycle #2 Against H. CoV
Test Cycle #1

| | Replicate 1 | | | | Replicate 2 | | | | Replicate 3 | | | | Cytotoxicity | | | |
|---|---|---|---|---|---|---|---|---|---|---|---|---|---|---|---|---|
| Dilution | (Virus) | | | | (Virus) | | | | (Virus) | | | | Control | | | |
| 10⁻² | 0 | 0 | 0 | 0 | 0 | 0 | 0 | 0 | 0 | 0 | 0 | 0 | 0 | 0 | 0 | 0 |
| 10⁻³ | 0 | 0 | 0 | 0 | 0 | 0 | 0 | 0 | 0 | 0 | 0 | 0 | 0 | 0 | 0 | 0 |
| 10⁻⁴ | 0 | 0 | 0 | 0 | 0 | 0 | 0 | 0 | 0 | 0 | 0 | 0 | 0 | 0 | 0 | 0 |
| Log10 TCID₅₀ | ≤1.5 | | | | ≤1.5 | | | | ≤1.5 | | | | N/A | | | |
| Log10 TCLD₅₀ | N/A | | | | | | | | | | | | ≤1.5 | | | |
| Log | ≥3.0 | | | | ≥3.0 | | | | ≥3.0 | | | | N/A | | | |
| Reduction | | | | | | | | | | | | | | | | |

N/A = Not applicable

TABLE 15
Medical Waste Device Test Cycle #3 Against H. CoV
Test Cycle #1

| | Replicate 1 | | | | Replicate 2 | | | | Replicate 3 | | | | Cytotoxicity | | | |
|---|---|---|---|---|---|---|---|---|---|---|---|---|---|---|---|---|
| Dilution | (Virus) | | | | (Virus) | | | | (Virus) | | | | Control | | | |
| 10⁻² | 0 | 0 | 0 | 0 | 0 | 0 | 0 | 0 | 0 | 0 | 0 | 0 | 0 | 0 | 0 | 0 |
| 10⁻³ | 0 | 0 | 0 | 0 | 0 | 0 | 0 | 0 | 0 | 0 | 0 | 0 | 0 | 0 | 0 | 0 |
| 10⁻⁴ | 0 | 0 | 0 | 0 | 0 | 0 | 0 | 0 | 0 | 0 | 0 | 0 | 0 | 0 | 0 | 0 |
| Log₁₀ TCID₅₀ | ≤1.5 | | | | ≤1.5 | | | | ≤1.5 | | | | N/A | | | |
| Log₁₀ TCLD₅₀ | N/A | | | | | | | | | | | | ≤1.5 | | | |
| Log | ≥3.0 | | | | ≥3.0 | | | | ≥3.0 | | | | N/A | | | |
| Reduction | | | | | | | | | | | | | | | | |

N/A = Not applicable

### CONCLUSION

Each test cycle of the medical waste device sterilization of H. CoV was performed in the presence of a 5% organic load. The virus recovery, cytotoxicity, and cell viability controls met the criteria. There was no virus detected within the detectable range (Log₁₀>1.5) after each test cycle run using the medical waste device.

We claim:

1. A device for independent thermal processing of one to four containers of medical waste comprising:
a) a compartment having two to four thermal chambers, wherein each thermal chamber is capable of receiving a single container of medical waste for said independent thermal processing;
b) one or more heating sources for each thermal chamber to provide heat to the thermal chamber for the independent thermal processing of the one to four containers of medical waste at a maximum temperature of between about 300° F. to about 425° F.;
c) a closure lid for each thermal chamber having a latching or locking mechanism that is engaged prior to the initiation of the independent thermal processing of the container of medical waste;
d) one or more exhaust tubes connected in the top portion of each thermal chamber allowing for vending of gaseous and liquid byproducts generated by the independent thermal processing of the container of medical waste;
e) a valve attached to each exhaust tube for each thermal chamber to separate the gaseous byproducts from the liquid byproducts, wherein the separated gaseous byproducts and liquid byproducts are vented by separate tubing attached to the valve;
f) at least one UV system fitted to each gaseous byproducts tube to allow for the sterilization of gaseous byproducts generated during the independent thermal processing of the container of medical waste prior to vending from the device; and
h) a central controller to operate, monitor, and control the device for the independent thermal processing of the one to four containers of medical waste, wherein the independent thermal processing of the one to four containers of medical waste results in the sterilization of the container of medical waste and gaseous byproducts.

2. The device of claim 1, wherein the compartment of the device has two thermal chambers.

3. The device of claim 2, wherein the two thermal chambers are able to receive containers of medical waste in the range of about 1 pint to about 4 gallons.

4. The device of claim 3, wherein the containers of medical waste range from about 1 pint to about 1 gallon.

5. The device of claim 1, wherein the medical waste is red bag biohazardous medical waste or sharps biohazardous medical waste.

6. The device of claim 1, wherein the one or more heating sources is an incorporated induction coil assembly.

7. The device of claim 1, wherein the independent thermal processing of the one to four containers of medical waste by the device comprises user activation of the device wherein a thermal chamber closure lid is latched or locked and a heating cycle is initiated for the thermal chamber containing the container of medical waste to heat the thermal chamber to a temperature of about 350° F. to about 400° F. for a period of about 60 minutes to about 90 minutes, after which the heater for the chamber is automatically turned off and the thermal processed container of medical waste cools to a safe temperature for handling and disposal.

8. The device of claim 7 wherein the thermal chamber is operated at about 350° F. for a period of 90 minutes.

9. The device of claim 7, wherein the heating cycle for the independent thermal processing of one to four containers of medical waste is initiated simultaneously or sequentially.

10. A process for independent thermal sterilization of one or more medical waste containers containing biohazardous medical waste for safe handling and disposal comprising a system for:
a) placing the one or more medical waste containers containing biohazardous medical waste into a device comprising two or more thermal chambers, wherein each thermal chamber can receive a single medical waste container and each thermal chamber is fitted with a lid with a latch or locking mechanism;
b) allowing the user to activate the device independently for one or more medical waste containers to be thermally processed;
c) the device activation, which initiates a process during which the lid to the thermal chamber containing the medical waste container to be thermally processed is latched or locked;
d) following the latching or locking of the lid by the device, a heating cycle is automatically initiated for the thermal chamber containing the medical waste to be processed, during which the thermal chamber is heated to a maximum temperature ranging from about 300° F. to about 425° F.;
e) the maximum temperature of the thermal chamber is automatically maintained for about 30 minutes to about 90 minutes; and
f) the heater for the thermal chamber is automatically turned off and the thermal chamber cools to a temperature of about 120° F. and the latched or locked lid is automatically unlatched or unlocked to allow for the safe removal and disposal by the user of the thermally processed medical waste container containing the sterilized biohazardous medical waste.

11. The process of claim 10, wherein the device comprises two thermal chambers.

12. The process of claim 11, wherein the thermal chambers are able to receive a single medical waste container in the range of about 1 pint to about 4 gallons.

13. The process of claim 12, wherein the single medical waste container ranges from about 1 pint to about 1 gallon.

14. The process of claim 10, wherein the medical waste container holds red bag biohazardous medical waste or sharps biohazardous medical waste.

15. The process of claim 10, wherein the maximum temperature ranges from about 350° F. to about 400° F. and the maximum temperature is maintained for a time ranging from about 60 minutes to about 90 minutes.

16. The process of claim 10, wherein the medical waste container holds a test load of red bag biohazardous medical waste comprising Staphylococcus aureus, Pseudomonas aeruginosa, Candida albicans, Aspergillus brasiliensis, Mycobacterium terrae, Mycobacterium hassiacum, Bacillus atrophaeus, and/or Geobacillus stearothermophilus, and the maximum temperature of the thermal chamber is about 350° F. which is maintained for a period of about 90 minutes.

17. The process of claim 10, wherein the medical waste container holds a test load of sharps biohazardous medical waste comprising Staphylococcus aureus, Pseudomonas aeruginosa, Candida albicans, Aspergillus brasiliensis, Mycobacterium terrae, Mycobacterium hassiacum, Bacillus atrophaeus, and/or Geobacillus stearothermophilus, and the maximum temperature of the thermal chamber is about 350° F. which is maintained for a period of about 90 minutes.

18. The process of claim 10, wherein the biohazardous medical waste container holds a test load of human coronavirus medical waste and the maximum temperature of the thermal chamber is about 350° F. which is maintained for a period of about 90 minutes.

19. A thermal process for sterilization of up to two medical waste containers holding bacterial and/or viral biohazardous medical waste in a device comprised of two thermal chambers, wherein each thermal chamber is capable of receiving a single medical waste container holding bacterial and/or viral biohazardous medical waste and wherein the thermal process for sterilization is carried out independently for the up-to-two medical waste containers by initiating a heating cycle with a maximum temperature of about 350° F. that is maintained for a period of about 90 minutes.

20. The process of claim 19, wherein the bacterial biohazardous medical waste comprises Staphylococcus aureus, Pseudomonas aeruginosa, Candida albicans, Aspergillus brasiliensis, Mycobacterium terrae, Mycobacterium hassiacum, Bacillus atrophaeus, and/or Geobacillus stearothermophilus.

21. The process of claim 20, wherein the viral biohazardous contaminated medical waste is comprised of human coronavirus.
